(19) United States
(12) Patent Application Publication
(10) Pub. No.: US 2010/0106027 A1
(43) Pub. Date: Apr. 29, 2010

(54) DEVICE FOR DETECTION AND ANALYSIS OF VITAL PARAMETERS OF THE BODY, SUCH AS, IN PARTICULAR, PULSE AND RESPIRATION

(76) Inventor: Robert Jaeger, Gondelsheim (DE)

Correspondence Address: COLLARD & ROE, P.C., 1077 NORTHERN BOULEVARD, ROSLYN, NY, 11576, US

(21) Appl. No.: 12/450,782

(22) PCT Filed: Apr. 17, 2008
(86) PCT No.: PCT/EP2008/003102

§ 371 Date: Oct. 13, 2009

(30) Foreign Application Priority Data
Apr. 18, 2007 (DE) 10 2007 018 677.2

(51) Int. Cl. A61B 5/0205

(52) U.S. Cl. 600/484

(57) ABSTRACT

The present invention relates to a device for detecting and analyzing vital parameters, particularly the pulse and respiration. A metal plate (3) fixed in direct proximity to the body surface (14) serves as a sensor element. The metal plate (3) is coupled to a non-linear LC oscillating circuit (11). Changes in the outermost body layers (15) caused by respiration and/or the pulse result in a shift in the resonant frequency of the LC oscillating circuit (11), in turn causing a change in the amplitude of oscillation. The amplitude of oscillation is measured, digitized, analyzed by a microprocessor (18), and evaluated. The result is displayed in a form understandable even to a layperson. The device operates automatically and determines the vital parameters independently. All components, including the power supply, are integrated in a small housing (2).

The present invention relates to a device with which vital parameters of the human body, such as pulse and respiration, can be detected and analyzed.

In industrial countries, illnesses in the cardiovascular system are by far the most frequent cause of death. In Germany alone, about 44% of all persons die of these causes. Sudden heart failure, in other words an acute failure of cardiovascular function, is suffered by approximately ten percent of the population. In this connection, there are many different reasons that lead to such a critical state. A cardiac infarction, severe injuries in an accident, or an electrical shock can lead to loss of consciousness that causes a respiratory and cardiac failure. In such situations, it is vital for the patient that his life-threatening condition be recognized immediately, and that he be adequately resuscitated without delay. With every minute without cardiopulmonary resuscitation (CPR), the likelihood of survival decreases by ten percent. Ten minutes after cardiovascular failure, there is normally no longer any chance of survival.

In order to be prepared for such everyday situations, it is particularly important that everyone be prepared and trained in the fundamental measures in emergency situations that involve unconscious patients. In this connection, aside from heart-lung resuscitation, it is particularly important to know how consciousness and the vital parameters are checked.

Since there are no obligatory courses in First Aid measures other than when getting a driver's license, and essential knowledge is often lost, accident witnesses trust themselves to help only in rare cases.

Another problem is the fact that resuscitation guidelines change at certain intervals. The most recent change in the guidelines of the “[in English:] European Resuscitation Council” took place in 2005. According to these guidelines, First Aiders merely have to check for consciousness and respiration. Before 2005, the pulse also had to be checked by feeling the carotid artery. The reasons why checking the pulse is no longer considered to be useful are the lack of competence and the lack of courage of the First Aiders.

There are already some suggestions for solving these problems.

The U.S. Pat. No. 4,958,638 “[in English:] Non-contact vital signs monitor” (Sharpe et al., 1990) describes a system with which pulse and respiration can be measured at the same time, without any physical contact of the electrodes or sensors with the body. High-frequency waves in the range of 10 GHz are emitted to the body surface, and the running time differences between the wave going out and coming back are measured or calculated. In this manner, data concerning respiration and pulse as well as all other movements at the body surface can be obtained.

The U.S. Pat. No. 3,993,995 “[in English:] Respiration Monitor” (Kaplan et al., 1976) describes a system for detecting respiration, without producing any direct body contact of the sensor. A part of the chest region is irradiated with light. The reflected light is detected. The phase difference between the emitted and reflected light is determined in a quadrature detector, which indirectly contains respiratory movements.

The U.S. Pat. No. 5,727,549 (Suda et al., 1998) proposes the following method: By means of pressing on a “[in English:] carbon fiber felt” its direct current resistance changes. As a result, a constant current through the material experiences a differently great resistance. If the system is glued onto the body surface, it experiences pressure changes as the result of the body movements brought about by respiration and pulse.

In the U.S. Pat. No. 5,273,036 (Kronberg et al., 1993), a method is described that utilizes reflection pulse-oximetry as a basis. Light-emitting diodes transmit light into the tissue. Photodetectors measure the reflected light, which is damped by the arteries. This changes as the result of the fluctuating pulse waves. If the sensor is adapted in the chest region, then the different intensity is also dependent on the respiratory movement and the compression of the tissue and the blood vessels that is triggered by this movement.

The U.S. Pat. No. 6,823,739 (Ueno et al., 2004) describes a system that can recognize point-like movement detection, such as respiration or pulse, by means of thin piezo material.

In the patent document U.S. Pat. No. 6,491,647 (Bridger et al., 2002), a method is described with which physiological parameters such as pulse and respiration, among others, can be measured in non-invasive and point-like manner. The system is based on mechanical changes of a measurement transducer that converts these changes to electrical signals. For this purpose, the measurement structure must be pressed down onto the body, for example by means of chest belts, so that the movements at the skin surface are transferred to the measurement transducer.

In the U.S. Pat. No. 6,875,176 (Mourad et al., 2005), a method is described in which movements at the body surface are detected by means of ultrasound. In this way, respiration and pulse can be detected, in addition to internal cranial pressure.

The system according to U.S. Pat. No. 6,758,816 (Tsuhata et al., 2004) takes advantage of the Doppler effect for determining the pulse. A transmitter sends an ultrasound wave into the artery; a receiver receives the reflected signal.

None of the previously known systems for detecting pulse and/or respiration have proven themselves in practice, since they are based on a more or less complicated and relatively expensive method. In the case of some of the methods described, a counter-pressure is required, which is produced by means of belts. Such systems are complicated in operation, are neither small nor useful to lay persons, and are therefore hardly suitable for mobile First Aid use. All the known devices furthermore require a relatively large amount of electrical energy, and this is not acceptable, particularly for mobile First Aid use.

In view of the disadvantages of the state of the art as has been shown, it is the task of the present invention to make available a system for the diagnosis of vital parameters such as respiration and pulse, which works quickly and reliably, and can also be used by inexperienced First Aiders at the accident site.

This task is accomplished by means of a device having the characteristics indicated in claim 1.

The device according to the invention is based on the idea of detecting the slightest changes in the uppermost body layers, and thereby recognizing the vital parameters, particularly pulse and respiration. Lifting and lowering of the ribcage or abdomen during respiration, just like the blood pulsing in the arteries, lead to periodic mechanical changes at the body surface. An LC oscillation circuit is used to detect these changes in the uppermost body layers, and a metallic sensor element is coupled with it. The sensor element is brought into the immediate vicinity of the body surface, without touching it. The sensor element represents an antenna for electromagnetic waves in the immediate area, or, in a first approximation, can be interpreted as a plate of a plate capacitor, whereby the adjacent body layers represent the other plate. Considered somewhat more precisely, however, the arrangement corresponds more to a spherical capacitor having a metallic sensor element in the middle, and the adjacent body layers as a dielectric. Even the slightest changes in the uppermost body layers or the slightest changes in the distance between sensor element and body surface thus bring about a change in capacitance, thereby causing the resonance frequency of the LC oscillation circuit to be raised or lowered slightly. De-tuning of the resonance circuit leads to a decrease in the oscillation amplitude. This voltage drop can . . . as direct voltage . . . ation amplitude. This voltage drop can be measured very simply, as direct voltage. The amplitude voltage measured at the LC oscillation circuit thus represents a measure of the mechanical changes in the uppermost skin layers. The vital parameters can be determined by means of analysis of the amplitude signal, using a microprocessor, and in particular, it can be recognized whether respiration and/or pulse is/are present.

Respiration and pulse of a human being can be recognized most easily at the neck, preferably in the region above the collarbone. In this connection, the sensor element does not necessarily have to be applied above the carotid artery, because the changes in the uppermost skin layers are sufficiently great, even at some distance, because of the propagation of the pulse wave into the surrounding tissue, so that they can be detected by the sensor element. Respiratory movements can be registered well all the way into the region of the shoulder.

It is a great advantage of the invention that the vital parameters of pulse and respiration are jointly detected at one location of the body. Placement of a single sensor element that can be structured to be small and light, at an easily accessible location of the body, such as, in particular, in the region of the neck or collarbone, makes the system particularly useful in an emergency and easy to handle even for lay persons. Another great advantage consists in that the system works automatically, in that it automatically analyzes the signal obtained from the changes in the uppermost body layers, and determines the vital parameters from this. The user therefore does not need to have any experience in feeling for a pulse, or to be trained in observing respiration. The most important question that a First Aider must ask himself/herself, namely whether or not the patient needs to be resuscitated, is quickly and clearly answered by the device. Nowadays, self-learning methods and algorithms are available for this, with which the detected signals can be analyzed and evaluated. In the simplest case, the evaluation unit can output unambiguous information, such as “Pulse and respiration present” or “Resuscitate immediately!”, for example.

The system works all the more accurately and reliably the more sensitively the LC oscillation circuit reacts to an influence on its resonance frequency as the result of capacitative, inductive, or even damping effects. For this reason, an LC oscillation circuit that has a non-linear characteristic with a very steep resonance curve is preferably used. In contrast to a linear oscillation circuit, in the case of a non-linear oscillation circuit having a very steep or actually an over-hanging resonance curve, even slight or extremely slight changes in inductance or capacitance lead to a disproportionately great change in the oscillation amplitude. Even the slightest changes in the uppermost skin layers can therefore be reliably detected. Another advantage when using a non-linear LC oscillation circuit is the very low energy consumption of the highly sensitive measurement system. The entire device can therefore be built to be very small and also cost-advantageous, and this is important for mobile use in rescue efforts.

Other advantageous and practical embodiments of the device according to the invention are evident from the dependent claims.

Exemplary embodiments of the invention will be explained in greater detail using the attached figures. These show:

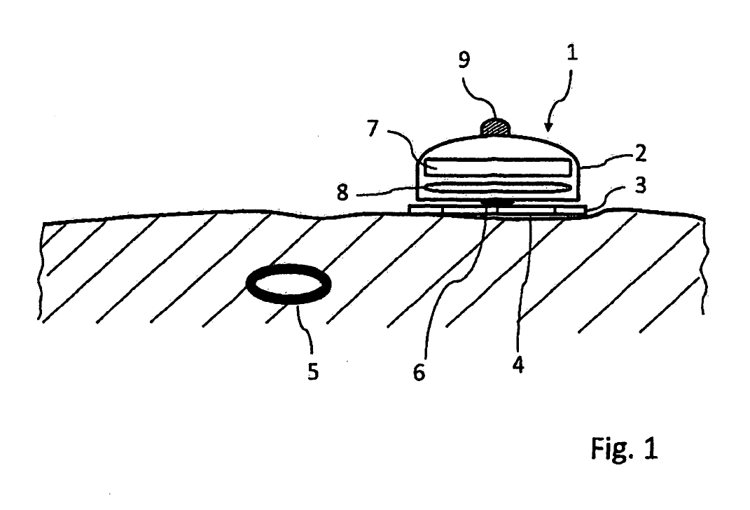

FIG. 1 a device for determining the vital parameters, greatly simplified;

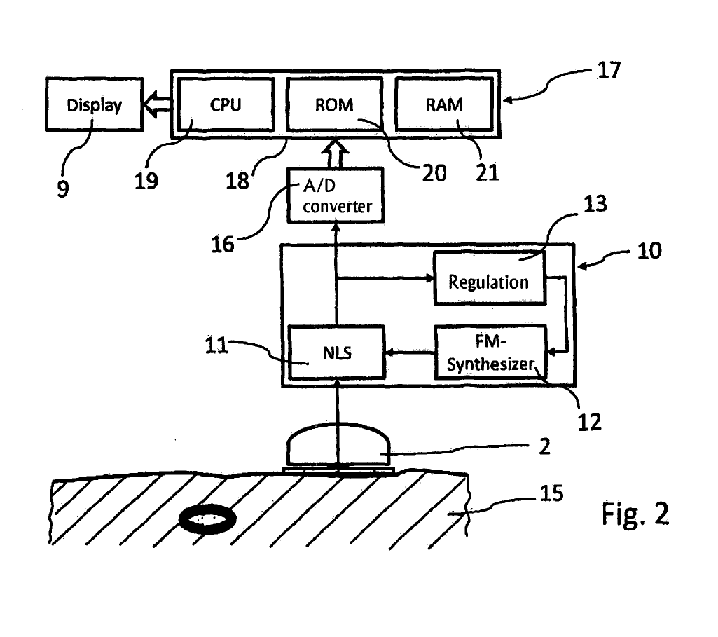

FIG. 2 a block schematic of the device of FIG. 1;

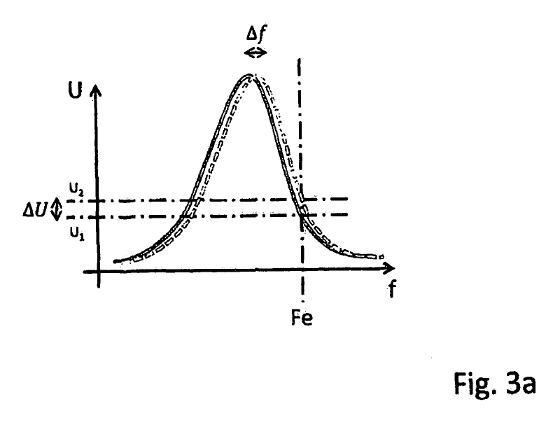

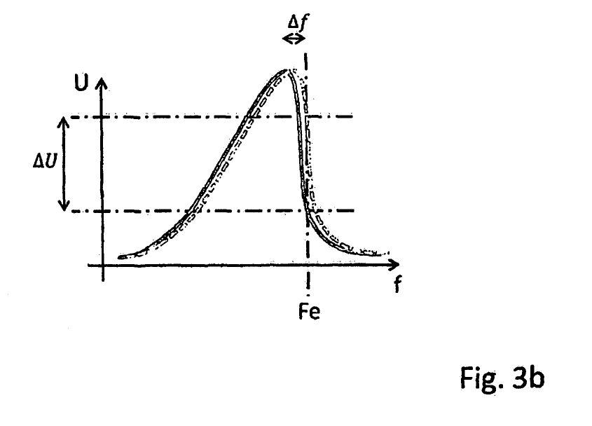

FIG. 3a the resonance curve of a linear LC oscillation circuit;

FIG. 3b the resonance curve of a non-linear LC oscillation circuit;

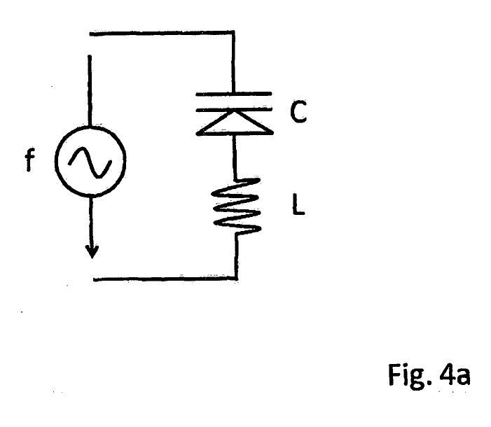

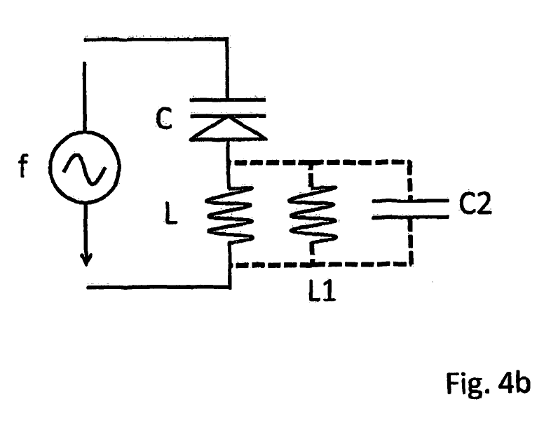

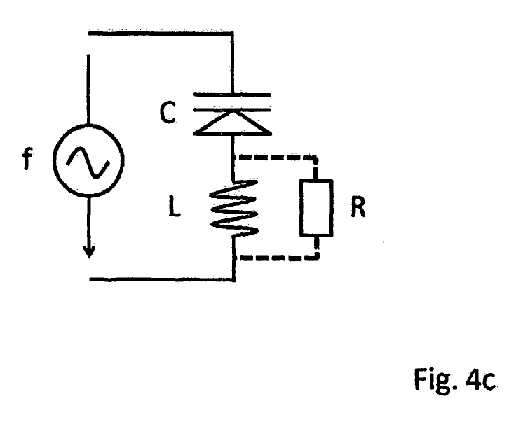

FIGS. 4a, 4b, 4c fundamental schematics of the non-linear LC oscillation circuit, with and without a load;

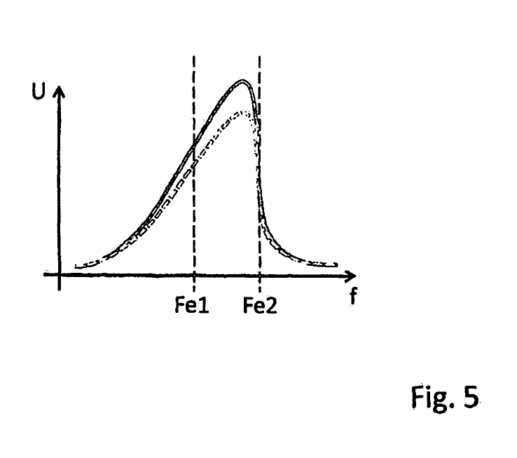

FIG. 5 the resonance curve of the non-linear oscillation circuit with and without ohmic loss resistance;

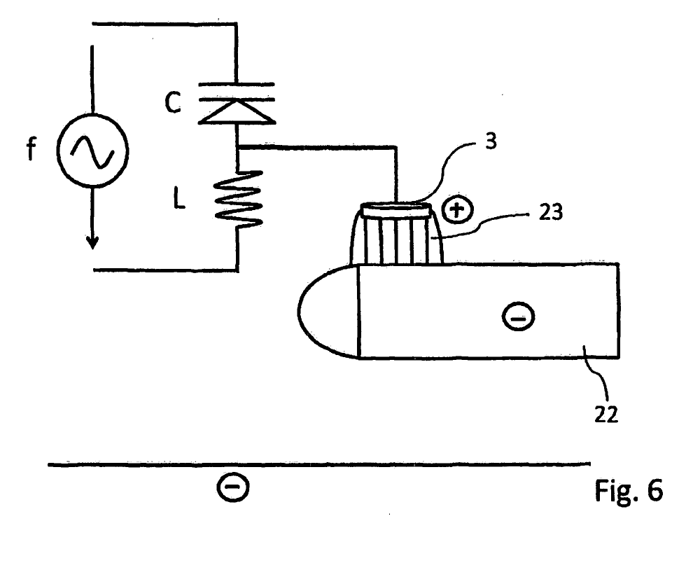

FIG. 6 the fundamental schematic of the non-linear LC oscillation circuit with the sensor element connected;

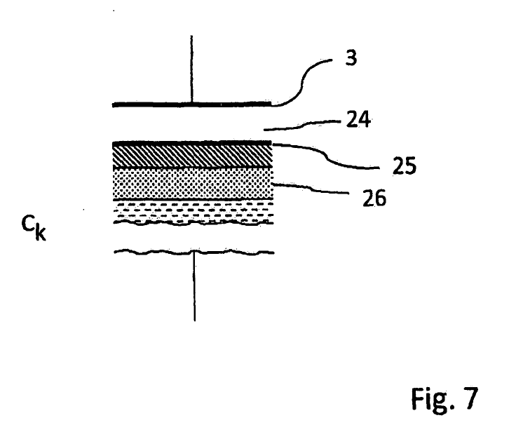

FIG. 7 a capacitor fundamental schematic of the sensor element brought close to the body surface;

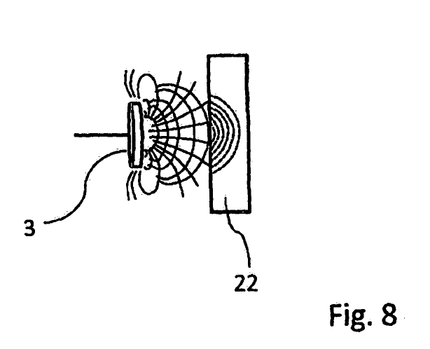

FIG. 8 the eddy current effects in the region of the sensor element;

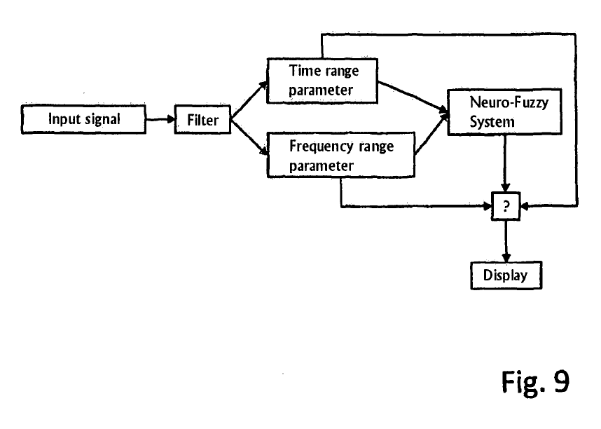

FIG. 9 a process chart for signal processing;

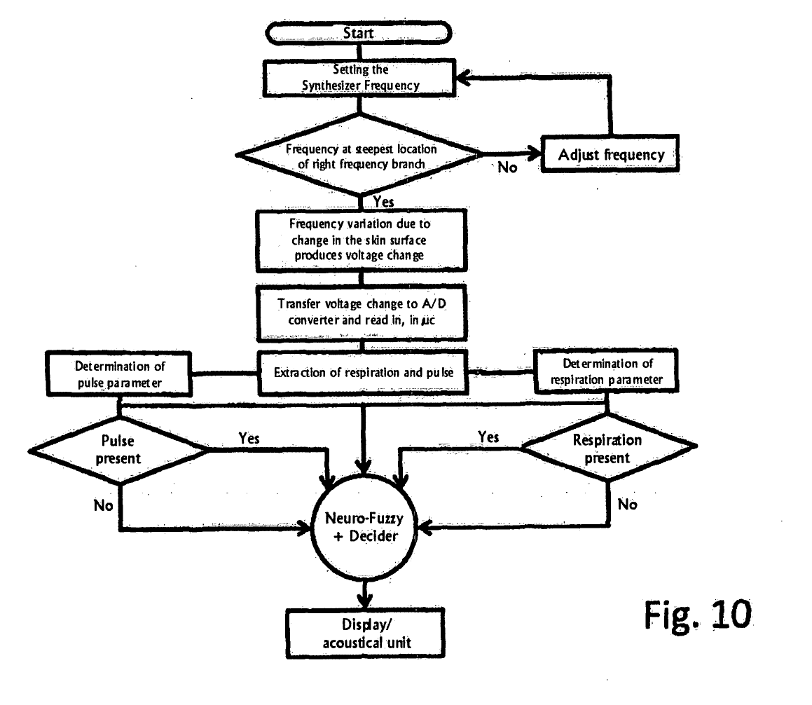

FIG. 10 a flow chart for the entire device;

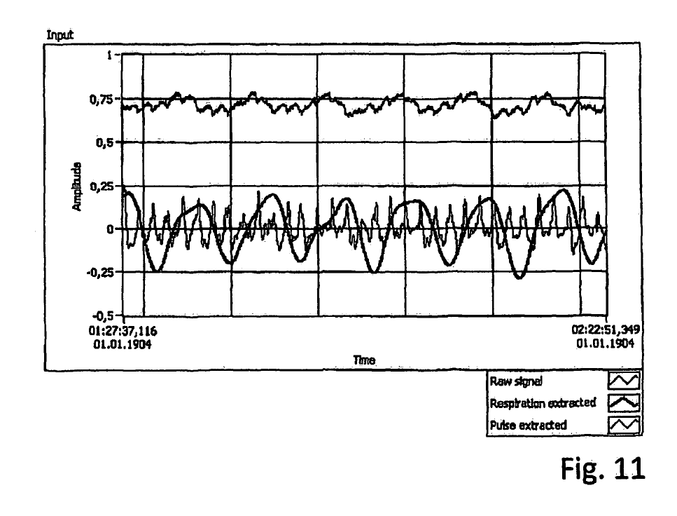

FIG. 11 an amplitude/time diagram for the measured raw signal and the signals for pulse and respiration extracted from it.

In FIG. 1, a device 1 especially intended for use in First Aid can be seen. All the electrical components are accommodated in a small, round housing 2. On the underside, there is a sensor element in the form of a thin metal plate 3, which is well conductive electrically. On its front (at the bottom, in the figure), the metal plate 3 is provided with a self-adhesive layer 4, which simultaneously has an electrical insulation layer and the function of a spacer. The device 1 is fixed in place on the patient's skin by means of the self-adhesive layer 4, specifically in the vicinity of the carotid artery 5. The thickness of the insulating layer is approximately 0.1 mm, and thereby defines the distance between metal plate 3 and body surface. On the back (at the top, in the figure), the metal plate 3 carries a plug connector 6, which functions in principle like a pushbutton, and which serves for mechanical and electrical connection with the components of the device built into the housing 2.

In the interior of the housing 2, there is a highly integrated electronic circuit 7 and an energy supply in the form of a battery 8. The result of the diagnosis is output, as an optical signal, on a display 9 that is affixed on the outside of the housing 2.

The block schematic of FIG. 2 shows the fundamental structure of the electronic circuit 7. It comprises a signal detection unit 10 having a non-linear LC oscillation circuit (NLS) 11, which is excited by a frequency generator 12. A frequency regulation 13 keeps the oscillation frequency that is set constant. The metal plate 3, which is fixed in place at a slight distance (approximately 0.1 mm) above the body surface 14 is coupled with the LC oscillation circuit 11 in such a manner that slight and the slightest movements, i.e. mechanical changes in the uppermost body layers 15 bring about a shift in the resonance frequency of the LC oscillation circuit 11. The change in oscillation amplitude of the LC oscillation circuit 11 resulting from this (while the excitation remains unchanged) is measured and is available at the output of the signal detection unit 10 as an analog voltage signal.

An analog/digital converter 16 converts the analog amplitude signal to a corresponding digital signal. The digitalized amplitude signal is passed to an evaluation unit 17, which consists of a microprocessor 18 having a CPU 19, non-volatile ROM memory 20 and RAM working memory 21. The microprocessor 18 analyzes the amplitude signal and determines, on the basis of criteria stored in memory, whether the patient is breathing sufficiently and/or has a sufficiently strong pulse. The result of the analysis is output as an optical and/or acoustical signal. The display 9 at the top of the housing 2 (see FIG. 1) serves for this purpose.

The LC oscillation circuit used here comprises not only an inductance (L) but also a non-linear capacitor diode, in other words a voltage-controlled capacitor (C) having a directional effect. Very steep resonance curves can be set with such a non-linear oscillation circuit. In the patent application DE 10 2005 010 498 of the same applicant, the method of functioning and the properties of a non-linear oscillation circuit are described in greater detail.

FIG. 3a illustrates the resonance curve of an LC oscillation circuit having a linear characteristic. The oscillation amplitude U is plotted above the frequency f. Changes in inductance or capacitance lead to a shift in the resonance frequency toward higher or lower frequencies. The resonance curve that has been shifted to the right in this example is shown with a broken line. If the oscillation circuit is excited at a frequency Fe, a shift in the resonance frequency by Δf leads to an approximately proportional drop in the oscillation amplitude ΔU.

FIG. 3b, in contrast, shows the resonance curve of a non-linear LC oscillation circuit. The very steep, almost vertical progression of the right branch of the resonance curve is noticeable. If one adjusts the exciter frequency Fe in such a way that it lies in the region of this steep curve branch, then even a slight shift in the resonance frequency Δf will already lead to a disproportional drop in the oscillation amplitude ΔU. Thus, extremely high amplitude changes can be produced with a non-linear LC oscillation circuit, without any great circuit technology effort. Changes in amplitude of almost any desired size can be implemented, even with the slightest changes in resonance frequency. In an extreme case, a resonance curve having a vertical flank or even an over-hanging resonance curve can actually be produced, so that the system tilts easily in a specific frequency range.

In FIG. 4a, the fundamental schematic of the non-linear LC oscillation circuit 11 from the circuit according to FIG. 2 is shown. The oscillation circuit contains not only the inductance L but also a varactor diode having the capacitor C. The resonance frequency is calculated according to the formula

$${f}_{0} = \frac{1}{2 π \sqrt{LC}}$$

In FIG. 4b, another inductance L1 and an additional capacitor C1 are switched in parallel to the inductance L of the oscillation circuit. In this way, the resonance frequency of the LC oscillation circuit is changed.

According to FIG. 4c, real resistors R can be added, in addition to inductive or capacitative components. However, their ohmic losses do not influence the resonance frequency of the oscillation circuit, but rather only the height and width of the resonance curve.

In FIG. 5, the typical resonance curve of a non-linear LC oscillation circuit is shown, once without and (with a broken line) with an additional ohmic loss resistance. If the exciter frequency Fe1 lies at a less steep location of the resonance curve, ohmic losses bring about a relatively great drop in oscillation amplitude. If the exciter frequency Fe2 lies in a very steep region of the resonance curve the same ohmic loss has a lesser effect as compared with the much greater collapse in amplitude as the result of a change in capacitance and/or inductance. Nevertheless, care must be taken to ensure that the resonance curve does not flatten completely as the result of overly great ohmic losses.

FIG. 6 shows the fundamental schematic of the non-linear LC oscillation circuit with the metal plate 3 connected between inductance L and capacitor C as a sensor element (see FIG. 1 and FIG. 2), which responds to changes in the body tissue 22. At a sufficiently high frequency, the metal plate 3 acts as an antenna for electromagnetic waves in the vicinity, and, greatly simplified, at the same time represents the one plate of a plate capacitor whose other plate is the body tissue 22. When it comes close enough to the body tissue 22, directed field lines 23 form. A change in the distance between metal plate 3 and body tissue 22 leads to a change in capacitance, as do changes in the body tissue 22 itself.

In contrast to a capacitor, however, the electrical parameters of the body tissue cannot be unambiguously described. This is connected with the fact that the body surface is composed of different tissue layers. Since these tissue layers are only relatively weakly electrically conductive, the electromagnetic waves emitted by the metal plate 3 penetrate at least into the uppermost layers, and in doing so actually hit arteries in which blood is flowing.

The uppermost skin layers and the interstice between the insulated metal plate 3 and the body surface can also be represented as a capacitor having multiple layers switched one behind the other, with different dielectricity constants.

FIG. 7 shows a corresponding fundamental schematic, comprising a metal plate 3 that serves as a sensor element, the interstice 24 between metal plate 3 and skin surface 25, as well as the skin layers 26 that lie directly underneath the skin surface 25. Each of these layers has a different dielectricity constant, which are all part of the total capacitance Ck. Changes, particularly expansions of the individual skin layers 26, as well as a change in the distance between metal plate 3 and skin surface 25, lead to a change in the capacitance Ck and thus also to a change in the total capacitance in the oscillation circuit, which leads to an increase or decrease of its resonance frequency.

FIG. 8 illustrates how the ohmic losses in the LC oscillation circuit are influenced by eddy current effects in the body tissue 22. These losses have an effect on the height and width of the resonance curve. Inductive effects, on the other hand, can be ignored.

The amplitude signal made available by the signal detection unit 10 (see FIG. 2) must be processed further in the evaluation unit 17, in such a manner that a result that is understandable even to a lay helper is displayed. The general sequence is shown in FIG. 9.

The digital input signal is fed into a self-regulating filter. Subsequently, extraction of typical characteristics in the time and frequency range, and an evaluation by a self-learning neuro-fuzzy system take place. Finally, all the parameters are brought together in a decision unit, and the decision generated there is transmitted to a display. All the partial results of the individual computation steps and the end result are stored in memory with a time stamp. Decision-making takes place using criteria recommended by doctors. Because of the use of a neuronal fuzzy-logic network, it is possible to also permit differentiated results, such as, for example, “Possible respiratory failure” or “Very weak pulse.”

The flow chart of FIG. 10 reproduces the entire process when using the device.

All that has to be done manually is to affix the device, by means of gluing the sensor element onto the body surface. From this moment on, all the steps, in other words detection and analysis of the vital parameters, all the way to output of a result signal, proceed automatically. The helper merely has to make sure that the patient does not move too much.

Immediately after the sensor element is glued on, first the capacitances, inductives and ohmic losses in the system are determined, and, based on these values, the exciter frequency for the LC oscillation circuit is set to an optimal value, in other words to the steepest location of the resonance curve. Once the optimal setting has been found, all that has to be monitored subsequently is whether the actual frequency deviates from the reference frequency over an extended period of time. If necessary, the actual frequency is adjusted, i.e. adapted to changed conditions. In this way, uniformly great sensitivity and measurement accuracy are guaranteed.

The changes in the uppermost skin layers that are brought about by respiration and/or pulse lead to de-tuning of the oscillation circuit, which in turn brings about a change in the oscillation amplitude. These voltage changes are digitalized by means of an A/D converter, and passed to the microprocessor. There, the raw signal, which comprises all the body parameters detected, is divided up into the sectors of “respiration” and “pulse.” Using special algorithms, the parameters and the quality of the pulse and respiration are determined separately. All data such as “Respiration or pulse present” or “not present,” for example, are finally linked with one another and evaluated in a self-learning fuzzy logic. The overall assessment obtained from this is output on a display, in clear text and/or as an acoustical signal.

FIG. 11 illustrates how the signals for pulse and respiration (bottom) are extracted from the measured raw signal (top), by means of the computer. The pulse signal has a much higher frequency here than the respiration signal, and this is an indication that the patient is breathing and has a normal pulse. The separate further processing of the extracted signals takes place by means of specific algorithms.

### REFERENCE SYMBOLS

1 device
2 housing
3 metal plate
4 self-adhesive layer
5 carotid artery
6 plug connector
7 electronic circuit
8 battery
9 display
10 signal detection unit
11 LC oscillation circuit
12 frequency generator
13 frequency regulation
14 body surface
15 body layers
16 A/D converter
17 evaluation unit
18 microprocessor
19 CPU
20 ROM memory
21 RAM memory
22 body tissue
23 field Tines
24 interstice
25 skin surface
26 skin layers

1. Device for detection and analysis of vital parameters of the body, such as, in particular, pulse and respiration, having
an LC oscillation circuit (11) that is excited to produce oscillations in the range of its resonance frequency;
a metallic sensor element that can be fixed in place at a slight distance from the body surface and is coupled with the LC oscillation circuit (11) in such a manner that small changes in the uppermost body layers (15) bring about a displacement of the resonance frequency, thereby causing a change in the oscillation amplitude;
a signal detection unit (10) that measures the oscillation amplitude of the LC oscillation circuit (11), and makes it available as an amplitude signal;
an evaluation unit (17) having a microprocessor (18), which analyzes and evaluates the amplitude signal, in order to determine the vital parameters.

2. Device according to claim 1, wherein the LC oscillation circuit (11) has a non-linear characteristic with a very steep resonance curve.

3. Device according to claim 2, wherein the LC oscillation circuit (11) comprises a non-linear capacitor diode.

4. Device according to claim 1, wherein the LC oscillation circuit (11) is excited by a frequency generator (12) having a frequency that can be regulated.

5. Device according to claim 4, wherein the sensor element is a thin metal plate (3).

6. Device according to claim 5, wherein the metal plate (3) has an insulation layer.

7. Device according to claim 5, wherein a spacer made of insulating material is affixed to the front of the metal plate (3).

8. Device according to claim 5, wherein the metal plate (3) carries a plug connector (6) on its back side.

9. Device according to claim 5, wherein the sensor element has a self-adhesive layer (4) that is provided for fixation on the skin.

10. Device according to claim 1, wherein an analog/digital converter (16) is disposed between the signal detection unit (10) and the evaluation unit (17), which converter converts the analog amplitude signal into a digital signal.

11. Device according to claim 1, further comprising a display (9) for optical and/or acoustical output of the result determined by the evaluation unit.

12. Device according to claim 11, wherein the sensor element having the LC oscillation circuit (11), the signal detection unit (10), the evaluation unit (17), and the display (9) are integrated into a common housing (2).

13. Device according to claim 12, wherein the housing (2) has an essentially planar underside, in the region of which the sensor element is disposed.
